(19) United States
(12) Patent Application Publication
(10) Pub. No.: US 2001/0016044 A1
(43) Pub. Date: Aug. 23, 2001

(54) Acoustic wave sensor for detecting contact state between a valve and a valve seat for a vehicle

(76) Inventor: Deog Jae Lee, Kyoungki-do (KR)

Correspondence Address: BIRCH STEWART KOLASCH & BIRCH, PO BOX 747, FALLS CHURCH, VA, 22040-0747, US

(21) Appl. No.: 09/749,663

(22) Filed: Dec. 28, 2000

(30) Foreign Application Priority Data
Dec. 29, 1999 (KR) 1999-65083

(51) Int. Cl. A61F 11/06; G10K 11/16; H03B 29/00

(52) U.S. Cl. 381/86; 381/71.4

(57) ABSTRACT

The present invention is related to an acoustic wave sensor for detecting a contact state between a exhaust□intake valve and a valve seat of valve train for a vehicle engine and more particularly, to an acoustic wave sensor which can detect an acoustic wave generated from outside through a speaker inserted inside of a manifold and through which a displayer can display a degree of a contact state between a exhaust□intake valve and a valve seat of valve train for a vehicle engine, whereby an operating time can be reduced, a more ideal working environment can be created, and an efficient inspection process can be performed due to a simplified inspection process.

### BACKGROUND OF THE INVENTION

1. Field of the Invention

The present invention is related to an acoustic wave sensor for detecting a contact state between a exhaust□intake valve and a valve seat of valve train for a vehicle engine and more particularly, to an acoustic wave sensor which can detect an acoustic wave generated from outside through a speaker inserted inside of a manifold and through which a displayer can display a degree of a contact state between a exhaust□intake valve and a valve seat of valve train for a vehicle engine, whereby an operating time can be reduced, a more ideal working environment can be created, and an efficient inspection process can be performed due to a simplified inspection process.

2. Information Disclosure Statement

Generally, in the endurance test for an engine, a main factor to lower a performance of the engine is loss in the compression force. In most cases, the loss in the compression force is caused by a depraved contact state between the exhaust□intake valve and the valve seat comprised for valve train of a vehicle engine.

In the field, therefore, the following works are performed for inspecting a contact state between the valve and the valve seat.

First, a valve is disassembled from a cylinder head by removing a valve spring and other related elements.

Second, the valve on which a surface is plastered with red stamping ink evenly is rotated to contact with the valve seat after the valve is inserted into a valve guide of the cylinder head.

Last, after the valve is disassembled from the cylinder head, the entire surface of the valve seat is inspected with a naked eye whether the red stamping ink is printed evenly. This inspection process is performed repeatedly as many as a number of the cylinders.

However, when a contact state between the valve and the valve seat is inspected by the above manner, it has drawbacks that most of the cylinder heads and valves have to be disassembled and reliability of the inspection cannot be obtained since the inspection process is performed with the naked eye, resulting in a different judgment from a different inspector.

Therefore, it is an urgent to develop an apparatus to detect a degree of a contact state between valve and the valve seat without disassembly of the members and to inspect with reliability.

### SUMMARY OF THE INVENTION

Therefore, it is an object of the present invention to provide an acoustic wave sensor which can detect an acoustic wave generated from outside through a speaker inserted inside of a manifold and through which a displayer can display a degree of a contact state between a exhaust□intake valve and a valve seat of valve train for a vehicle engine, whereby an operating time can be reduced, a more ideal working environment can be created, and an efficient inspection process can be performed due to a simplified inspection process.

The present invention is described in detail as set forth hereunder.

The acoustic wave sensor according to the present invention comprises an acoustic wave generating means and an acoustic wave sensing means.

The acoustic wave generating means consists of an acoustic wave oscillator for generating an acoustic wave in response to an operation of a switch, a first amplifier for amplifying the acoustic wave of the acoustic wave oscillator, and a speaker for diverging the acoustic wave of the first amplifier,

The acoustic wave sensing means consists of an acoustic wave sensing part for sensing the acoustic wave diverged through the speaker and converting the acoustic wave into an electric signal, a second amplifier for amplifying the signal of the acoustic wave sensing part and a display part for displaying a signal output from the second amplifier.

### BRIEF DESCRIPTION OF THE DRAWINGS

For fuller understanding of the nature and objects of the invention, reference should be had to the following detailed description taken in conjunction with the accompanying drawing in which:

FIG. 1 is a schematic view of an acoustic wave sensor for detecting a contact state between a exhaust□intake valve and a valve seat of valve train for a vehicle engine according to the present invention.

### DETAILED DESCRIPTION OF THE INVENTION

The present invention comprises an acoustic wave generating means and an acoustic wave sensing means. The acoustic wave generating means consists of an acoustic wave oscillator 2 for generating an acoustic wave in response to an operation of a switch 1, a first amplifier 3 for amplifying the acoustic wave of the acoustic wave oscillator 2, and a speaker 5 for diverging the acoustic wave of the first amplifier 3. The acoustic wave sensing means consists of an acoustic wave sensing part 11 for sensing the acoustic wave diverged through the speaker 5 and converting the acoustic wave into an electric signal, a second amplifier 12 for amplifying a signal of the acoustic wave sensing part 11, and a display part 13 for displaying a signal output from the second amplifier 12. The present invention further comprises a sound shielding member 4 mounted to a port part 6 for preventing the acoustic wave from leaking.

Especially, the speaker 5 of the acoustic wave generating means is installed at a bending portion of a tubular passage 7, and the acoustic wave sensing part 11 of the acoustic wave sensing means is installed a site under a contact surface between a valve 8 and a valve seat 9.

Also, the acoustic wave sensing part 11 of the acoustic wave sensing means comprises a condenser microphone for sensing the acoustic wave.

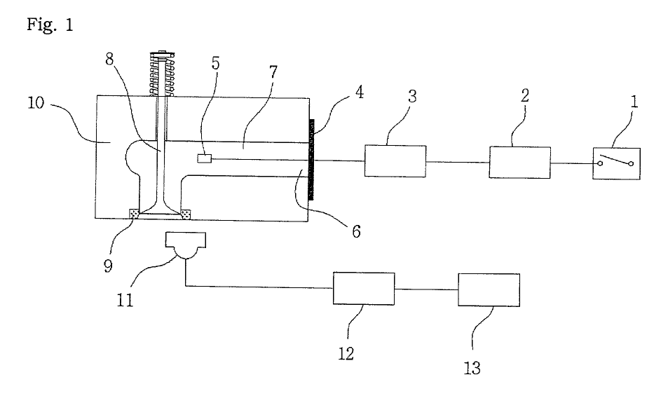

Hereinafter, the present invention will be described in greater detail. FIG. 1 is a schematic view of the acoustic wave sensor for detecting a contact state between the exhaust□intake valve and a valve seat of valve train for a vehicle engine according to the present invention. A reference numeral 10 indicates a cylinder body.

As shown in FIG. 1, the present invention is divided into of the acoustic wave generating means and the acoustic wave sensing means.

Also, in addition, the sound shielding member 4 is installed at certain location for preventing the acoustic wave from leaking.

The acoustic wave generating means is a means to generate the acoustic wave and treats and output the acoustic wave. The acoustic wave generating means consists of the acoustic wave oscillator 2 for generating the acoustic wave in response to an operation of the switch 1, the first amplifier 3 for amplifying the acoustic wave of the acoustic wave oscillator 2, and the speaker 5 for diverging the acoustic wave of the first amplifier 3.

Here, the switch 1 is a means for controlling a power supply and the acoustic wave oscillator 2 generates the acoustic wave artificially. It is preferable to use a lower frequency oscillator, which can be used easily, as the acoustic wave oscillator 2.

Furthermore, the first amplifier 2 amplifies a low frequency acoustic wave generated in the acoustic wave oscillator 2.

The switch 1, the acoustic wave oscillator 2 and the first amplifier 3 can be integrated into a single member and are installed outside of the port part 6.

The speaker 5 diverges the amplified acoustic wave output from the first amplifier 3 where the acoustic wave generated at the acoustic wave oscillator 2 is amplified. Said speaker 5 is installed at a bending portion of the tubular passage 7.

In order to prevent a leakage of the acoustic wave, the port part 6 is covered with the sound shielding member 4. It is desirable that the sound shielding member 4 is made of a material such as a conventional glass wool fiber.

On the other hand, the acoustic wave sensing means consists of the acoustic wave sensing part 11 for sensing the acoustic wave output through a contact surface between the valve 8 and the valve seat 9 toward which the acoustic wave diverged through the speaker 5 is output, the second amplifier 12 for amplifying a micro acoustic wave signal sensed by the acoustic wave sensing part 11 and the display part 13 for displaying an amplified signal through the second amplifier 12.

The acoustic wave sensing part 11 comprises the condenser microphone for sensing the acoustic wave by using a difference of the pressure between the acoustic waves. The condenser microphone will be described briefly in below.

A parallel cap with thickness of 50 μm is located at a very thin diaphragm and both polarities are opposed against each other so that an air condenser is formed. When a position of the diaphragm is changed in response to a pressure of the acoustic wave, a capacitance is changed in proportion to a displacement of the diaphragm. Therefore, the condenser microphone converts the capacitance into the electric signal.

On the other hand, the display part 13 displays the amplified signal through the second amplified 13 on a screen, whereby the operator can be find whether the acoustic waver is sensed or not. Also, the display part 13 preferably consists of a monitor using a liquid crystal display (LCD), etc.

In the acoustic wave sensor for detecting a contact state between the valve and the valve seat for the vehicle according to the present invention, the speaker to which an acoustic wave generated at an outside is input is mounted in a manifold and the sensing device senses whether the acoustic wave is existed or not at lower end of the valve and a display part displays a result to find a degree of a contact state between a valve and a valve seat. Therefore, the operating time can be reduced, a more ideal working environment can be created and a rapid inspection process can be performed due to a simplified inspection process

Although this invention has been described in its preferred form with a certain degree of particularity, it is appreciated by those skilled in the art that the present disclosure of the preferred form has been made only by way of example and that numerous changes in the details of the construction, combination, and arrangement of parts may be resorted to without departing from the spirit and scope of the invention.

What is claimed is:

1. An acoustic wave sensor for detecting a contact state between a exhaust□intake valve and a valve seat of valve train for a vehicle engine comprising an acoustic wave generating means and an acoustic wave sensing means.

2. The acoustic wave sensor for detecting a contact state between a exhaust□intake valve and a valve seat of valve train for a vehicle engine according to claim 1, wherein said acoustic wave generating means is consisted of an acoustic wave oscillator, a first amplifier for amplifying the acoustic wave of the acoustic wave oscillator, and a speaker for diverging the acoustic wave of the first amplifier.

3. The acoustic wave sensor for detecting a contact state between a exhaust□intake valve and a valve seat of valve train for a vehicle engine according to claim 1, wherein said acoustic wave sensing means is consisted of an acoustic wave sensing part for sensing an acoustic wave diverged through the speaker and converting the acoustic wave into an electric signal, a second amplifier for amplifying a signal of the acoustic wave sensing part and a display part for displaying a signal output from the second amplifier.

4. The acoustic wave sensor for detecting a contact state between a exhaust□intake valve and a valve seat of valve train for a vehicle engine according to any one of claims 1, 2 and 3, wherein a speaker is installed at a bending portion of a tubular passage, and said acoustic wave sensing part is installed a site under the contact surface between the valve and the valve seat.

5. The acoustic wave sensor for detecting a contact state between a exhaust□intake valve and a valve seat of valve train for a vehicle engine according to any one of claims 1 and 3, wherein said acoustic wave sensing part comprises a condenser microphone for sensing the acoustic wave.

6. The acoustic wave sensor for detecting a contact state between a exhaust□intake valve and a valve seat of valve train for a vehicle engine according to claim 4, wherein a sound shielding member, where said speaker is installed, is separately mounted to a port part for preventing the acoustic wave from leaking.
